(19) United States
(12) Patent Application Publication
(10) Pub. No.: US 2022/0153716 A1
(43) Pub. Date: May 19, 2022

(54) SYSTEM AND METHOD FOR THE EXTRACTION OF ISOFLAVONES FROM SOYBEANS

(71) Applicant: Rowan University, Glassboro, NJ (US)

(72) Inventors: John D. Chea, Sicklerville, NJ (US); Joseph F. Stanzione, III, Wilmington, DE (US); Kirti M. Yenkie, Glassboro, NJ (US)

(21) Appl. No.: 17/528,805

(22) Filed: Nov. 17, 2021

Related U.S. Application Data
(60) Provisional application No. 63/114,579, filed on Nov. 17, 2020.

(51) Int. Cl. C07D 311/36

(52) U.S. Cl. CPC C07D 311/36

(57) ABSTRACT

The present disclosure describes a system for the commercial scale extraction of isoflavones from soybean meal. The system includes an optional grinder, an extraction component, a solid-liquid separation component, an acid hydrolysis component, a neutralization component, and a purification component. Also provided are methods of commercial scale extraction of one or more isoflavones from soybean meal. The method includes extracting an isoflavone glucoside from soybean meal, hydrolyzing the isoflavone glucoside to obtain an isoflavone aglycone, neutralizing the isoflavone aglycone, and purifying the isoflavone aglycone.

### CROSS-REFERENCE TO RELATED APPLICATIONS

This application claims the benefit of priority to U.S. Provisional Patent Application Ser. No. 63/114,579 entitled “SYSTEM AND METHOD FOR THE EXTRACTION OF ISOFLAVONES FROM SOYBEANS,” filed Nov. 17, 2020, the disclosure of which is incorporated herein by reference in its entirety.

### STATEMENT REGARDING FEDERALLY SPONSORED RESEARCH OR DEVELOPMENT

This invention was made with government support under W911NF-14-2-0086 awarded by the U.S. Army and NP96259218 awarded by the Environmental Protection Agency. The government has certain rights in the invention.

### BACKGROUND

The United States has produced over 125 million metric tons of soybean annually, and approximately 55 million metric tons are exported to other countries. Of the remaining quantity of soybean grown in the U.S., 97% is used in the animal feed industry. Soybean meal is rich in nutrients such as essential amino acids, proteins, and carbohydrates. However, it also contains phytoestrogens such as isoflavones, which have no nutritional value and can affect the physiological and pathological processes related to livestock reproduction, bone remodeling, skin, and cardiovascular and immune systems upon excess consumption. Conversely, if these isoflavones were extracted from the soybean meal prior to animal consumption, any adverse effects on animals can be prevented and the extracted isoflavones can be used for commercial purposes. Although isoflavones content in soybean meal is low, the immense volumes of soybean meal produced yearly could be directed to produce significant volumes of high added value bio-based chemicals and materials while also recycling high protein soybean meal back to the animal feed supply chain.

There is an existing global market for isoflavones worth 2.9 billion (USD) that is expected to reach 50.06 billion (USD) by 2025. Soy isoflavone is becoming increasingly popular in the health and nutraceuticals sector for their anti-inflammatory and cancer inhibition effects in humans. Isoflavones present in soybean meal also have characteristics that are desirable in polymer design and performance. As per the U.S. soybean statistics, about 40 million metric tons of soybean meal is available for isoflavone extraction per year; however, the commercial process for isoflavone extraction does not exist today.

There is a need in the art for a commercially scalable system and method to extract isoflavones from soybeans. The present invention satisfies this unmet need.

### BRIEF SUMMARY OF THE INVENTION

In one aspect, a system for commercial scale extraction of an isoflavone from soybean meal is provided. The system includes: an optional grinder, an extraction component, a solid-liquid separation component, an acid hydrolysis component, a neutralization component, and a purification component, wherein the system extracts about 500 to about 1000 kg//h of soybean meal.

In one aspect, a method of commercial scale extraction of one or more isoflavones from soybean meal is provided. The method includes the steps of extracting an isoflavone-glucoside from soybean meal at a soybean meal feed rate of about 500 to about 1000 kg//h, hydrolyzing the isoflavone glucoside to obtain an isoflavone aglycone, neutralizing the isoflavone aglycone, and purifying the isoflavone aglycone.

Surprisingly and advantageously, the systems and methods described herein can produce as much as 1 kg/h of isoflavone or isoflavone aglycone, including the isoflavones genistein, daidzein, and glycitein.

### BRIEF DESCRIPTION OF THE DRAWINGS

The following detailed description of exemplary embodiments of the invention will be better understood when read in conjunction with the appended drawings. For the purpose of illustrating the invention, non-limiting embodiments are shown in the drawings. It should be understood, however, that the invention is not limited to the precise arrangements and instrumentalities of the embodiments shown in the drawings.

FIG. 1 depicts a superstructure of isoflavone extraction and purification pathways wherein bypass (BYP) streams are used to skip optional separation stages that do not appreciably impact the outcome of the overall process.

FIG. 2 and FIG. 3 depict the assumptions made to arrive at the optimal isoflavone extraction pathway depicted in FIG. 12 and FIG. 13. Additional assumptions are detailed in the experimental examples.

FIGS. 4-11 depict the optimal system components and overall process based on the assumptions detailed herein.

FIG. 12 and FIG. 13 depict the optimal extraction pathway based on the assumptions detailed herein.

FIG. 14 depicts the nutritional content of soybeans.

FIG. 15 depicts a schematic of the method for extracting isoflavones from soybean meal.

FIG. 16 depicts GAMS optimization.

FIGS. 17-19 depict the economic evaluation of the commercial scale isoflavone extraction.

### DETAILED DESCRIPTION OF THE DISCLOSURE

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, selected methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “soy bean” as used herein means the bean of the legume species Glycine max.

The term “soybean meal” as used herein means the solid residue left after oil extraction from soybeans. Soybean meal is frequently used as animal feed. Soybean meal can be de-fatted soybean meal without soybean hulls, de-fatted soybean meal with soybean hulls, or a combination thereof.

### Description

The present disclosure provides in one aspect a system for the extraction of one or more isoflavones from soybean meal. The present disclosure further provides a method for the extraction of one or more isoflavones from soybean meal. In some embodiments, the systems and methods disclosed herein can be used for the commercial scale extraction of one or more isoflavones from soybean meal. The skilled artisan will understand that the system components and method steps disclosed herein can be replaced with interchangeable components known to a person of skill in the art. The skilled artisan will further understand that the optimal systems and/or pathways disclosed herein can change with changes in material and energy balances, design options, utilities, cost, and/or industrial constraints. The skill artisan will further understand that GAMS optimization and/or other mathematical models may be used to determine the optimal systems and/or pathways for the extraction of isoflavones from soybean meal.

### Systems

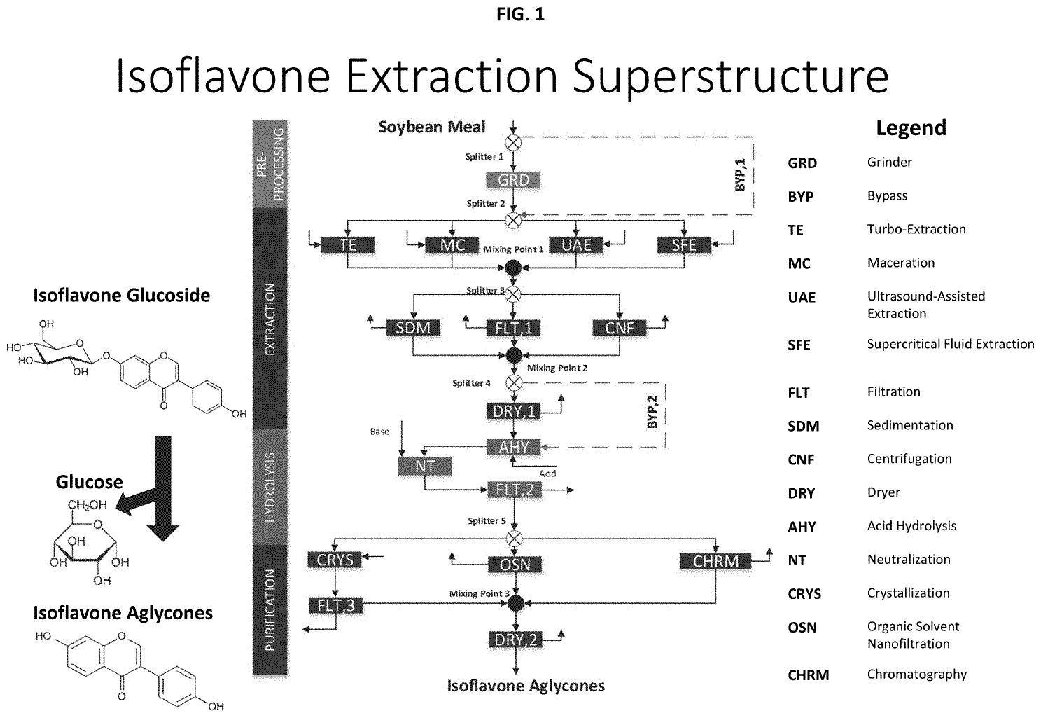

In one aspect, the present disclosure relates to a system for the extraction of one or more isoflavones from soybean meal (FIG. 1). In one embodiment, the system is used for the commercial extraction of one or more isoflavones from soybean meal. In some embodiments, the system can sustain a soybean meal feed flow rate of about 1000 kg/hr, or about 500 kg/hr to about 3000 kg/hr, with a yearly operating hour of about 7920 hrs or about 330 workdays. In various embodiments, the soybean meal feed rate is at least, greater than, or equal to about 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, or 3000 kg/hr. In various embodiments, the soybean meal feed rate is about 100 to about 1000, 200 to about 1000, 300 to about 1000, 400 to about 1000, 500 to about 1000, or 600 to about 1000 kg/hr.

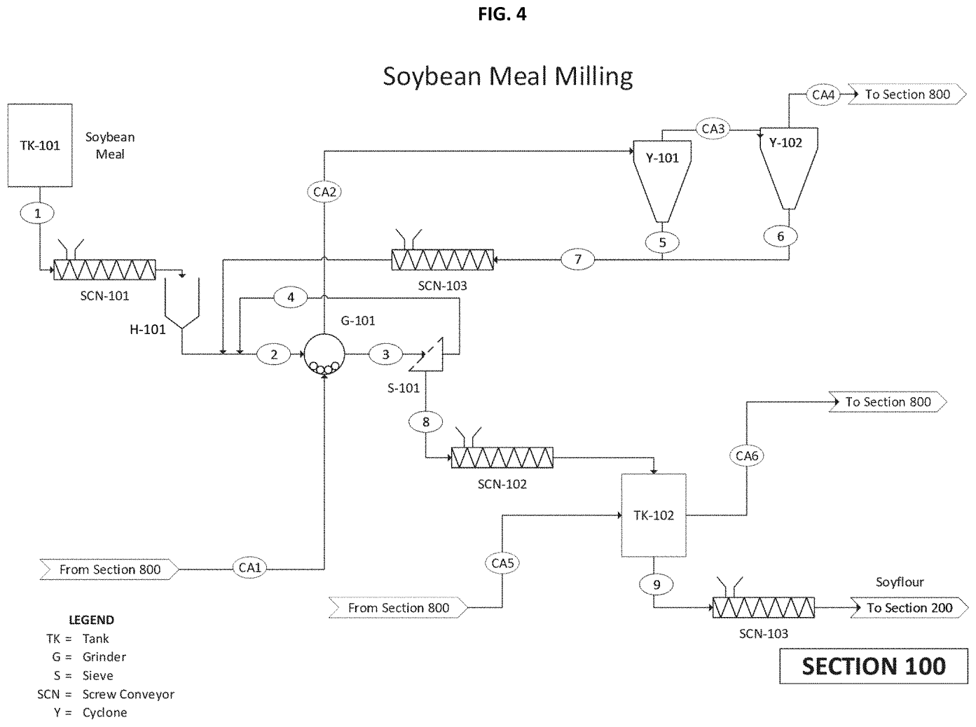

### Soybean Meal Milling (Section 100, FIG. 4)

Referring to the process flow diagram in FIG. 4, in one embodiment the system comprises a soybean meal milling portion (Section 100), that includes: a first soybean meal storage tank (TK-101), a first screw conveyor (SCN-101), a hopper (H-101), a grinder (G-101), a sieve (S-101), a second screw conveyor (SCN-102), a second screw conveyor (SCN-103), a second storage tank (TK-102), a first cyclone (Y-101), and a second cyclone (Y-102). The numbered ovals in FIG. 4, numbered 1 through 9, represent conduits that can transport solids, liquids, gases/vapors and mixtures of solids, liquids, and gases/vapors between the indicated components in FIG. 4, as well as other system sections as described herein.

In various embodiments, the soybean meal (primary feed) has a density of about 721 kg/m³ and a heat capacity of about 1.45 kJ/kg ° C. In various embodiments, cooling air (CA) air exists only in CA1, CA2, CA3, CA4, CA5, and CA6. In various embodiments, CA1 entering G-101 helps prevent overheating and degradation of soy particles during the grinding process. In various embodiments, the stream CA2 contains air and residual soybean flour that were swept out of the grinder. I various embodiments, Y-101 and Y-102 are cyclone units that separate air from soybean flour according to density. In various embodiments, SCN-103 conveyed the collected solid back to rejoin stream 2 and re-enters G-101.

In various embodiments, sieve S-101 rejects soybean meal particles that are greater than about 5 to about 500 microns. In various embodiments, sieve S-101 rejects soybean meal particles that are greater than about 250 microns. Stream 4 contains rejected soybean meal with particle size greater than about 250 microns. Below 250 microns size, soybean meal is considered “soybean flour”. In various embodiments, SCN-102 conveyed soybean flour to a holding tank (TK-102). Cooling air from stream CA5 is used to provide proper ventilation and maintain soybean flour at room temperature before the extraction in Section 200.

In one embodiment, the system optionally contains a grinder (GRD). In one embodiment, the optional grinder grinds soybean meal into a particle size between about 5 μm to about 500 μm. In one embodiment, the optional grinder grinds soybean meal into a particle size between about 5 μm to about 450 μm. In one embodiment, the optional grinder grinds soybean meal into a particle size between about 5 μm to about 400 μm. In one embodiment, the optional grinder grinds soybean meal into a particle size between about 5 μm to about 350 μm. In one embodiment, the optional grinder grinds soybean meal into a particle size between about 5 μm to about 300 μm. In one embodiment, the optional grinder grinds soybean meal into a particle size between about 5 μm to about 250 μm. In one embodiment, the optional grinder grinds soybean meal into a particle size between about 5 μm to about 200 μm. In one embodiment, the optional grinder grinds soybean meal into a particle size between about 5 μm to about 150 μm. In one embodiment, the optional grinder grinds soybean meal into a particle size between about 5 μm to about 100 μm. In one embodiment, the optional grinder grinds soybean meal into a particle size between about 5 μm to about 75 μm. In one embodiment, the optional grinder grinds soybean meal into a particle size between 25 μm to about 65 μm. In various embodiments, the grinder grinds the soybean meal into a particle size of at least, greater than, or equal to about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500 μm. The particle size can be an average particle size as determined by measuring the largest dimension of the soybean meal particle. Particle sizes can be determined by art-recognized techniques such as dynamic light scattering, static light scattering, or laser diffraction using equipment known in the art to be suitable for this purpose.

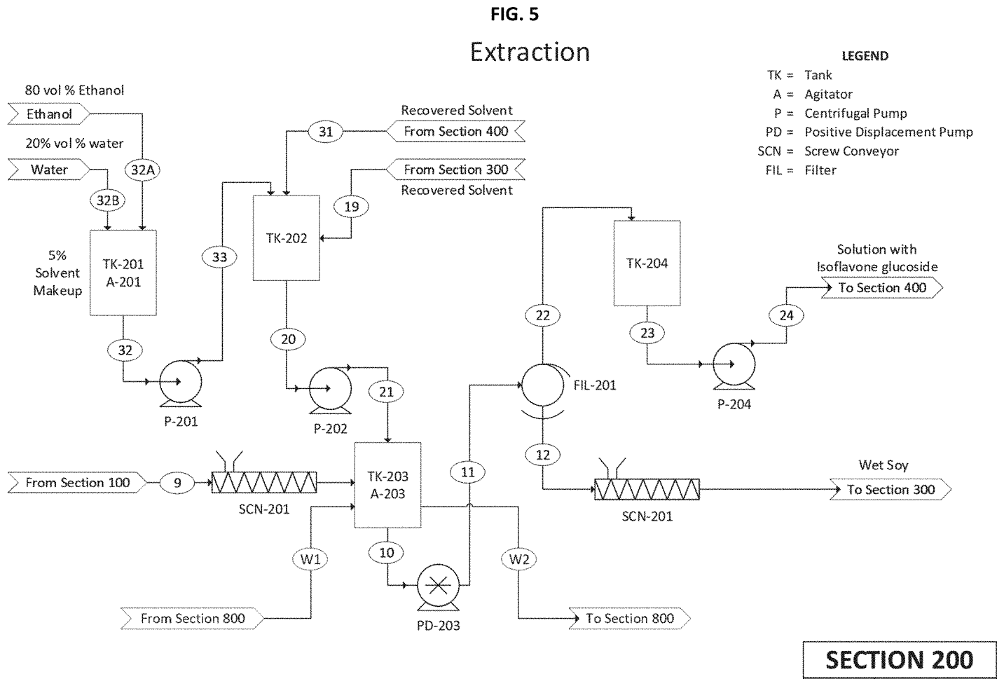

### Soybean Meal Extraction (Section 200, FIG. 5)

Referring to the process flow diagram in FIG. 5, in one embodiment the system comprises a soybean meal extraction portion (Section 200), that includes: a first storage tank (TK-201 ), agitator (A-201) in storage tank TK-201, a first centrifugal pump (P-201), a second centrifugal pump (P-202), a third centrifugal pump (P-204), a second storage tank (TK-202), a third storage tank (TK-203), a fourth storage tank (TK-204), a second agitator (A-203) in third storage tank TK-203, a positive displacement pump (PD-203), a filter (FIL-20 ), a first screw conveyor (SCN-201), and a second screw conveyor (SCN-201). The numbered ovals in FIG. 5, numbered 32A, 32B, 32, 33, 31, 19, 20, 21, 10, 11, 12, 22, 23, 24, W1, and W2, represent conduits that can transport solids, liquids, gases/vapors and mixtures of solids, liquids, and gases/vapors between the indicated components in FIG. 5, as well as other system sections as described herein. In various embodiments, the system and method of Section 200 is a turbo extraction (TE) system and method.

In various embodiments, the soybean meal enters the extraction stage through Stream 9, where it is conveyed into a mixing tank (TK-203) with an agitator (A-203).

In various embodiments, at steady state, ethanol and water are mixed in TK-201 with agitator A-201 in a separate instance, which served as a 5% make-up. The content of TK-201 served as a fresh solvent reservoir to ensure consistent flow. Solvents from this point forward refers to the combination of water and ethanol.

In various embodiments, the second storage tank (TK-202) collects recovered solvents from Section 300 and 400. The content of TK-202 is pumped into TK-203 and is mixed with the soybean flour. Positive displacement pump (PD-203) transported solid (soybean flour) and liquid (extracted isoflavone glucoside and solvent) to a vacuum filter (FIL-201). Solid soybean flours exit as Stream 12 and conveyed through a screw conveyor (SCN-201) to Section 300 for drying. The extracted materials and solvent exit the filter in Stream 22 and placed in holding tank TK-204. Pump (P-204) transports the mixture to Section 400.

The system comprises an extraction component selected from a turbo-extraction (TE) component, a maceration (MC) component, an ultrasound-assisted extraction (UAE) component, or supercritical fluid extraction (SFE) component. The extraction component contacts the soybean meal with one or more solvents to extract one or more isoflavone glucosides from the soybean meal. In one embodiment, the extraction occurs at room temperature (about 25° C.). In some embodiments, the extraction does not break down the soybean meal so that it may be recycled for use as animal feed following isoflavone glucoside extraction.

In one embodiment, the extraction component is a TE component, a MC component or a UAE component and the solvent comprises a mixture of an alcohol and water. In some embodiments, the extraction solvent comprises between about 50% and about 95% of an alcohol and between about 5% and about 50% water. In one embodiment, the extraction solvent comprises between about 95% and about 60% of an alcohol and between about 5% and about 40% water. In one embodiment, the extraction solvent comprises between about 95% and about 70% of an alcohol and between about 5% and about 30% water. In one embodiment, the extraction solvent comprises between about 85% and about 70% of an alcohol and between about 15% and about 40% water. In one embodiment, the extraction solvent comprises between about 85% and about 75% of an alcohol and between about 15% and about 30% water. In one embodiment, the extraction solvent comprises between about 85% and about 75% of an alcohol and between about 15% and about 25% water. In some embodiments, the extraction solvent comprises about 80% of an alcohol and about 20% water. In one embodiment, the alcohol is ethanol. In one embodiment, the extraction component is a TE component. In various embodiments, the solvent contains a ratio of alcohol:water of about 95:5, 90:10, 85:15, 80:20, 75:25, 70:30, 65:35, 60:40, 55:45, 50:50, 45:55, 40:60, 35:65, 30:70, 25:75, 20:80, 15:85, 10:90, or about 5:95.

In one embodiment, the extraction component is a SFE component and the extraction solvent comprises an alcohol and supercritical carbon dioxide. In one embodiment, the alcohol is ethanol.

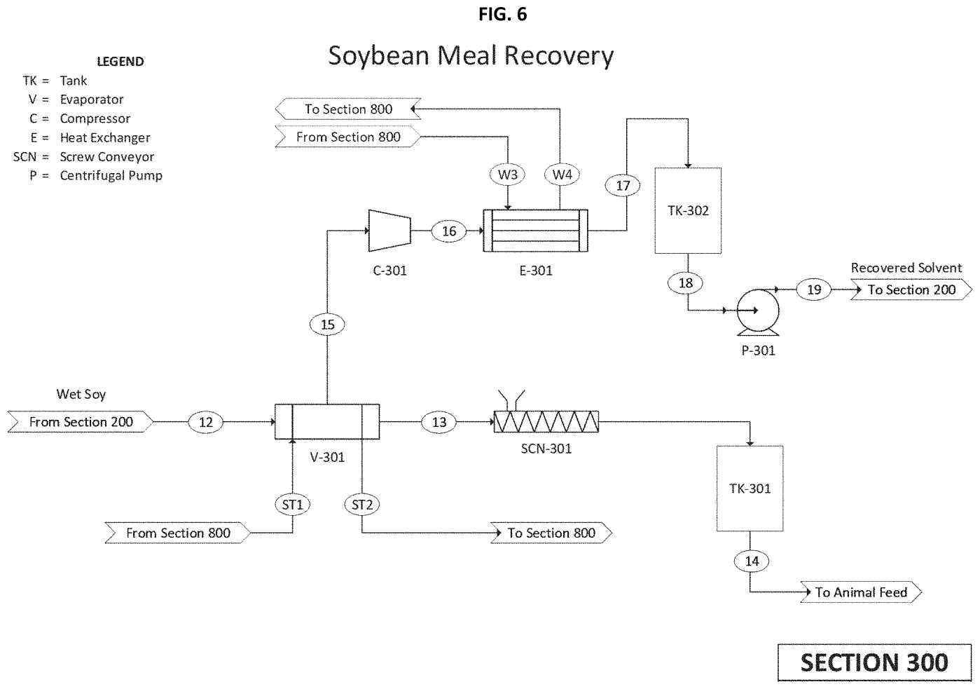

### Soybean Meal Recovery (Section 300, FIG. 6)

Referring to the process flow diagram in FIG. 6, in one embodiment the system comprises a soybean meal recovery portion (Section 300, that includes: an evaporator (V-301), a compressor (C-301), a heat exchanger (E-301), a screw conveyor (SCN-301), a first storage tank (TK-301), a second storage tank (TK-302), and a centrifugal pump (P-301). The numbered ovals in FIG. 6, numbered 12, 15, 16, 13, W3, W4, 17, ST1, ST2, 18, 19, and 14, represent conduits that can transport solids, liquids, gases/vapors and mixtures of solids, liquids, and gases/vapors between the indicated components in FIG. 6, as well as other system sections as described herein.

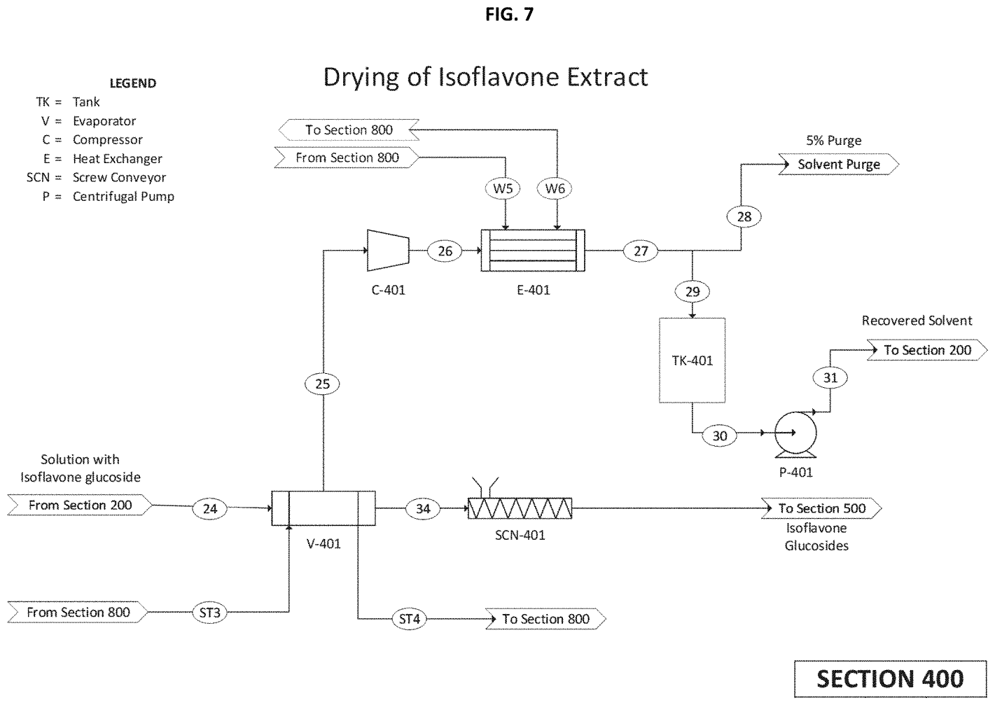

### Drying Isoflavone Extract (Section 400, FIG. 7)

Referring to the process flow diagram in FIG. 7, in one embodiment the system comprises a soybean meal recovery portion (Section 400), that includes: an evaporator (V-401), a compressor (C-401), a heat exchanger (E-401), a screw conveyor (SCN-401), a storage tank (TK-401), and a centrifugal pump (P-401). The numbered ovals in FIG. 7, numbered 24, ST3, 25, ST4, 34, 36, 27, 29, 28, 30, and 31, represent conduits that can transport solids, liquids, gases/vapors and mixtures of solids, liquids, and gases/vapors between the indicated components in FIG. 7, as well as other system sections as described herein.

In various embodiments, the isoflavone extract in solvent enters evaporator (V-401) through stream 24. ST3 and ST4 represents superheated steam designed to evaporate 99% of the solvents present. The dried isoflavone glucoside is conveyed through screw conveyor (SCN-401) in Stream 34 and exits to Section 500 for acid hydrolysis. The evaporated solvent exits V-301 through Stream 25. The vapor phase is compressed through C-401. Heat exchanger (E-401) is used to condense the compressed gas into liquid phase. The liquid is transported back to TK-202 from Section 200.

The system further comprises a solid-liquid separation component selected from a sedimentation (SDM) component, a filtration (FLT,1) component, and a centrifugation (CNF) component. The solid-liquid separation component separates the soybean meal from the one or more extraction solvents containing the extracted isoflavone-glucoside. In some embodiments, the separated soybean meal is used as animal feed. In some embodiments, the one or more extraction solvents are recovered (DRY,1) and can be reused. In one embodiment, the solid-liquid separation component is a filtration component.

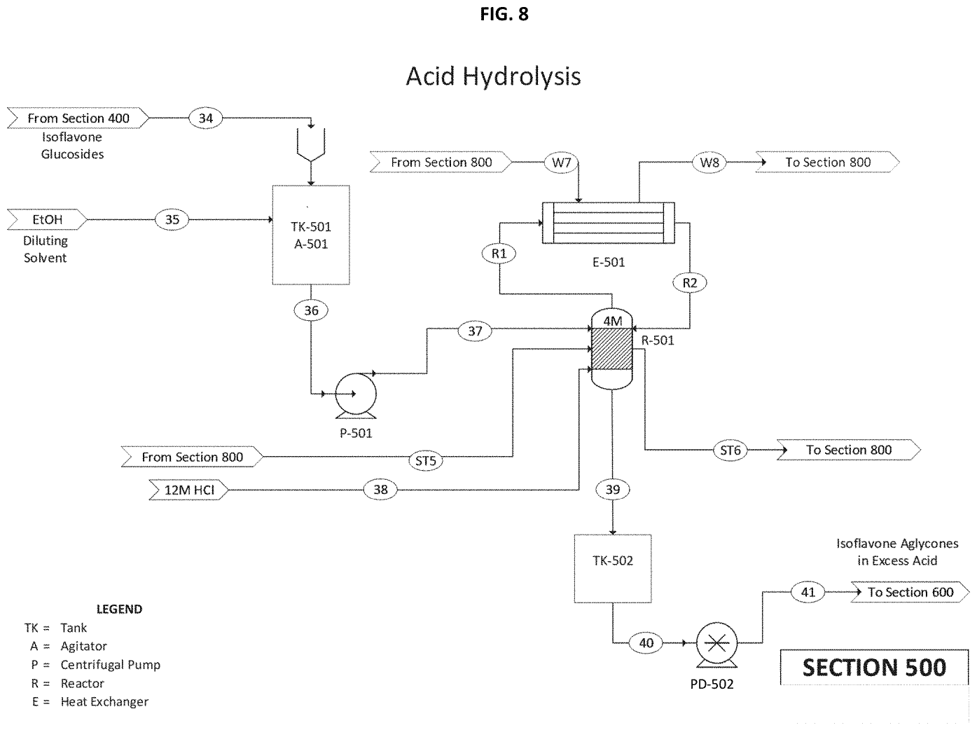

### Acid Hydrolysis (Section 500, FIG. 8)

Referring to the process flow diagram in FIG. 8, in one embodiment the system comprises an acid hydrolysis portion (Section 500), that includes: a first storage tank (TK-501), a second storage tank (TK-502), and agitator (A-501) in storage tank TK-501, a centrifugal pump (P-501), a reactor (R-501), a heat exchanger (E-501), and a positive displacement pump (PD-502). The numbered ovals in FIG. 8, numbered 34, 35, 36, 38, 37, R1, R2, 39, 40, ST6, and 41, represent conduits that can transport solids, liquids, gases/vapors and mixtures of solids, liquids, and gases/vapors between the indicated components in FIG. 8, as well as other system sections as described herein.

In various embodiments, isoflavone glucoside enters a mixing tank TK-501 with agitator A-501. Stream 35 is fed into the mixing tank to dilute the isoflavone glucoside before the reactor. The content of TK-501 is transported through pump P-501 into reactor R-501. Stream ST5 contains superheated steam to provide sufficient activation energy for the hydrolysis to occur. The vapor generated from heating the liquid content is sent to a heat exchanger designed for cooling (E-501). The liquid content is returned to the reactor R-501 at steady state. The content of the reactor contains isoflavone aglycones, excess hydrochloric acid, and dehydrated glucose molecules. Positive displacement pump (PD-502) transported the solid-liquid mixture to Section 600 for neutralization.

The system further comprises an acid hydrolysis (AHY) component wherein the isoflavone-glucoside undergoes an acid hydrolysis reaction to cleave the natural glucose attached to the isoflavone molecule. In one embodiment, the isoflavone-glucoside is contacted with an acidic solution. The acidic solution can comprise any acid known to a person of skill in the art. Exemplary acids include, but are not limited to, hydrochloric acid, sulfuric acid, nitric acid, acetic acid, citric acid, formic acid, carbonic acid, and combinations thereof. In one embodiment, a solution of hydrochloric acid is used for the acid hydrolysis. In one embodiment, the solution of hydrochloric acid has a molarity of between about 0.1 M and about 10 M. In one embodiment, the solution of hydrochloric acid has a molarity of between about 0.1 M and about 8 M. In one embodiment, the solution of hydrochloric acid has a molarity of between about 0.1 M and about 6 M. In one embodiment, the solution of hydrochloric acid has a molarity of between about 0.1 M and about 4.5 M. In one embodiment, the solution of hydrochloric acid has a molarity of between about 2.5 M and about 4.5 M.

In some embodiments, acid hydrolysis reaction occurs between an isoflavone glucoside dissolved/dispersed in an alcohol and a solution of hydrochloric acid in water, wherein the reaction occurs in a solution having a molarity of between about 2.5 M and about 4.5 HCl. In some embodiments, the acid hydrolysis reaction occurs at about room temperature. In another embodiment, the acid hydrolysis reaction occurs in the presence of steam to provide energy for hydrolysis. In various embodiments, the acidic solution has a concentration of at least about, greater than, or equal to about 0.5, 1, 1.5, 2, 2.5, 3, 3.5, 4, 4.5, 5, 5.5, 6, 6.5, 7, 7.5, 8, 8.5, 9, 9.5, or about 10 M in any of the acids mentioned herein.

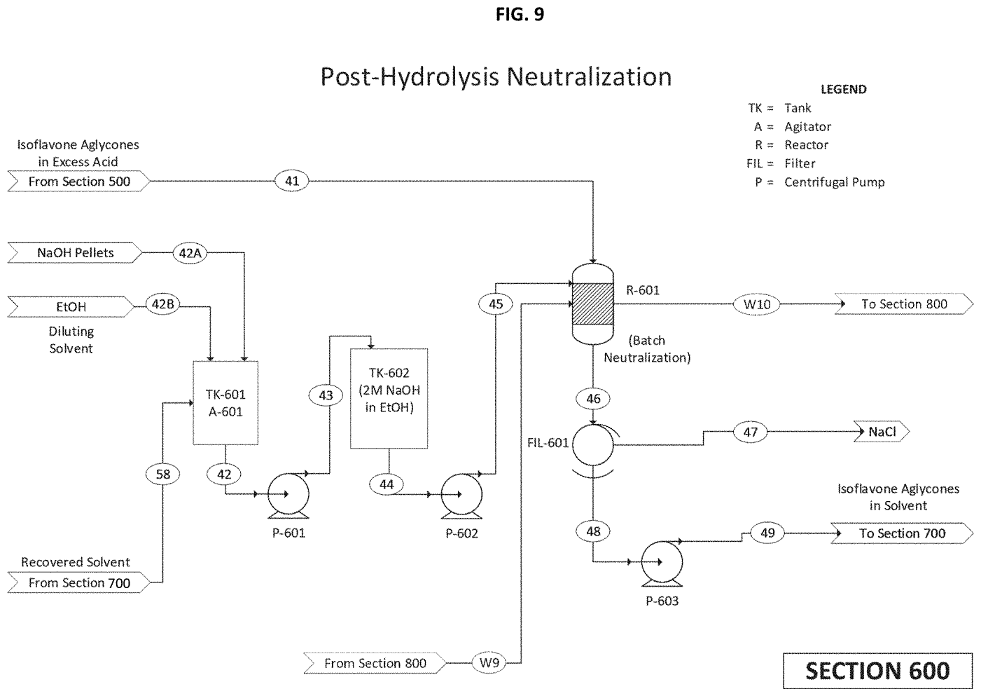

### Post-hydrolysis Neutralization (Section 600, FIG. 9)

Referring to the process flow diagram in FIG. 9, in one embodiment the system comprises post-hydrolysis neutralization portion (Section 600), that includes: a first storage tank (TK-601), an agitator (A-601) in storage tank TK-601, a first centrifugal pump (P-601), a second centrifugal pump (P-602), a third centrifugal pump (P-603), a second storage tank (TK-602), a reactor (R-601), and a filter (FIL-601). The numbered ovals in FIG. 9, numbered 41, 42A, 42B, 58, 42, 43, 44, 45, 46, 48, W10, 47, and 49, represent conduits that can transport solids, liquids, gases/vapors and mixtures of solids, liquids, and gases/vapors between the indicated components in FIG. 9, as well as other system sections as described herein.

In various embodiments, the isoflavone aglycones in excess acid enters another reactor R-601 in Stream 41. In a separate operation, sodium hydroxide (NaOH) pellets from Stream 42A and ethanol for dilution from Stream 42B are sent to a mixing tank TK-601 with agitator A-601. Recovered solvents from Section 700 is added to TK-601 to prepare a base solution of approximately 2M NaOH in TK-602. The NaOH in ethanol is transported to reactor R-601 through pump P-602. Cooling water W9 is used as a heat transfer medium to prevent excessive heat build-up.

In various embodiments, salt (NaCl) is expected to precipitate out during neutralization because it is insoluble in organic solvent. Vacuum Filter FIL-601 is used to separate NaCl from the product. However, the residual salt may remain in solution phase because the reaction of hydrochloric acid and sodium hydroxide generates water. The isoflavone aglycones product is transported by pump P-603 into Section 700 for purification.

The system further comprises a neutralization (NT) component wherein a neutralization reaction occurs as the acid hydrolysis solution is contacted with a base. Suitable bases include, but are not limited to, LiOH, NaOH, KOH, Na₂CO₃, NaHCO₃, aqueous NH₃, NH₄Cl, NH₄OH, and the like. In one embodiment, the hydrolysis solution is contacted with a solution comprising a base. In one embodiment, solution comprising a base has a molarity of between about 0.1 M and about 10 M. In one embodiment, the solution comprising a base has a molarity of between about 0.1 M and about 8 M. In one embodiment, the solution comprising a base has a molarity of between about 0.1 M and about 6 M. In one embodiment, the solution comprising a base has a molarity of between about 0.1 M and about 4 M. In one embodiment the solution comprising a base has a molarity of between about 1 M and about 3 M. In some embodiments, the base is sodium hydroxide (NaOH). In one embodiment, a 2 M solution of sodium hydroxide in ethanol is used in the NT component to neutralize the acid hydrolysis solution. In some embodiments, the NT component further comprises a filtration (FLT,2) component to remove precipitated salt formed during the neutralization reaction. In various embodiments, the basic solution has a concentration of at least about, greater than, or equal to about 0.5, 1, 1.5, 2, 2.5, 3, 3.5, 4, 4.5, 5, 5.5, 6, 6.5, 7, 7.5, 8, 8.5, 9, 9.5, or about 10 M in any of the bases mentioned herein.

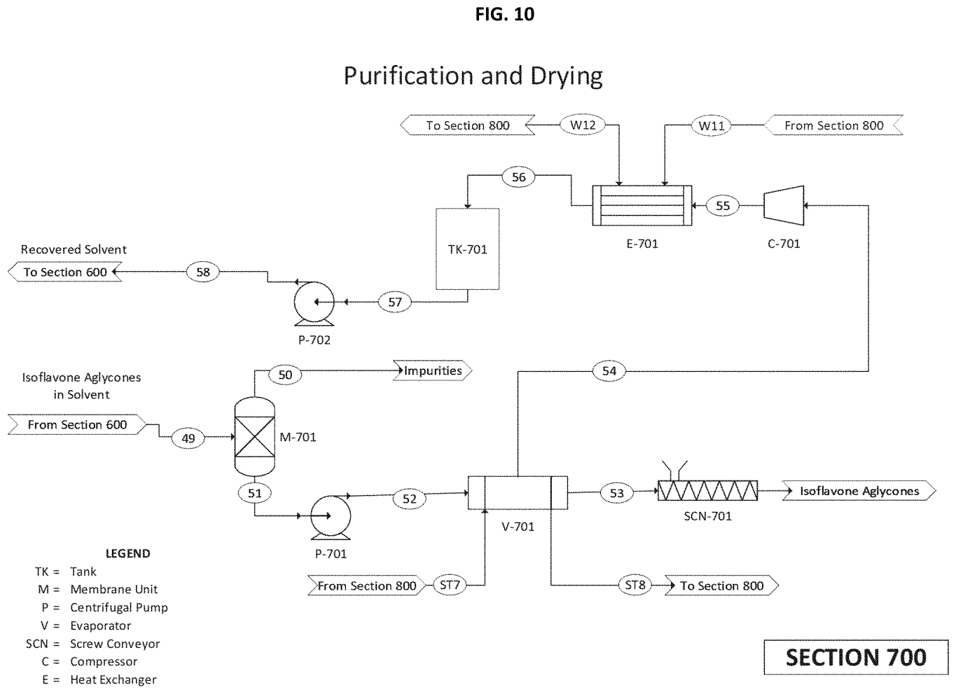

### Isoflavone Purification and Drying (Section 700, FIG. 10)

Referring to the process flow diagram in FIG. 10, in one embodiment the system comprises an isoflavone purification and drying portion (Section 700), that includes: a first centrifugal pump (P-701), a second centrifugal pump (P-702), membrane unit (M-701), a storage tank (TK-701), an evaporator (V-701), a heat exchanger (E-701), a compressor (C-701), and a screw conveyor (SCN-701). The numbered ovals in FIG. 10, numbered 58, 49, 57, 50, 51, 52, 56, 54, 53, ST7, ST8, 55, W11, and W12 represent conduits that can transport solids, liquids, gases/vapors and mixtures of solids, liquids, and gases/vapors between the indicated components in FIG. 10, as well as other system sections as described herein.

In various embodiments, the product stream (Stream 49) is sent into a membrane unit M-701 to remove impurities (Stream 50). Salt (NaCl), dehydrated sugar, carbons, and other impurities are removed. In various embodiments, the purified product is transported to evaporator V-701 by pump P-701. Superheated steam (ST7) is used to evaporate residual solvent. The final product is transported by screw conveyor (SCN-701) in Stream 53.

The evaporated solvent exits V-701 through Stream 54. The vapor phase is compressed through C-701. Heat exchanger (E-701) is used to condense the compressed gas into liquid phase. The liquid is collected in tank TK-701 and transported to Section 600 (neutralization) as Stream 58 to be used for base dilution.

The system further comprises a purification component selected from a crystallization (CRYS) component, an organic solvent nanofiltration (OSN) component, or a chromatography (CHRM) component to remove remaining impurities from the isoflavone aglycone product. In one embodiment, an OSN component is selected to remove the remaining impurities. In some embodiments, an antisolvent is added to solution from the neutralization reaction to force the nonpolar isoflavone to precipitate out of the solution. In one embodiment, the antisolvent is water. In one embodiment, the precipitated purified isoflavone is removed from the solution using the OSN component.

In some embodiments, the purification component further comprises a drying (DRY,2) component to remove traces of solvent from the final isoflavone aglycone product.

In some embodiments, General Algebraic Modeling Systems (GAMS) optimization is used to select the optimal system components to extract an isoflavone-glucoside from soybean meal (i.e. TE, MC, UAE), the optimal system components to separate the soybean meal from the isoflavone-glucoside (i.e. SDM, FLT,1, CNF), and the optimal system components to purify the isoflavone aglycone (i.e. CRYS, OSN, CHRM). In one embodiment, GAMS optimization uses Mixed-Integer Nonlinear Programing (MINLP) wherein material and energy balances, design options, utilities, cost, and/or industrial constraints are considered to provide a recommended set of separation and purification technologies required to extract and isolate isoflavone aglycones from soybean meal at the commercial scale.

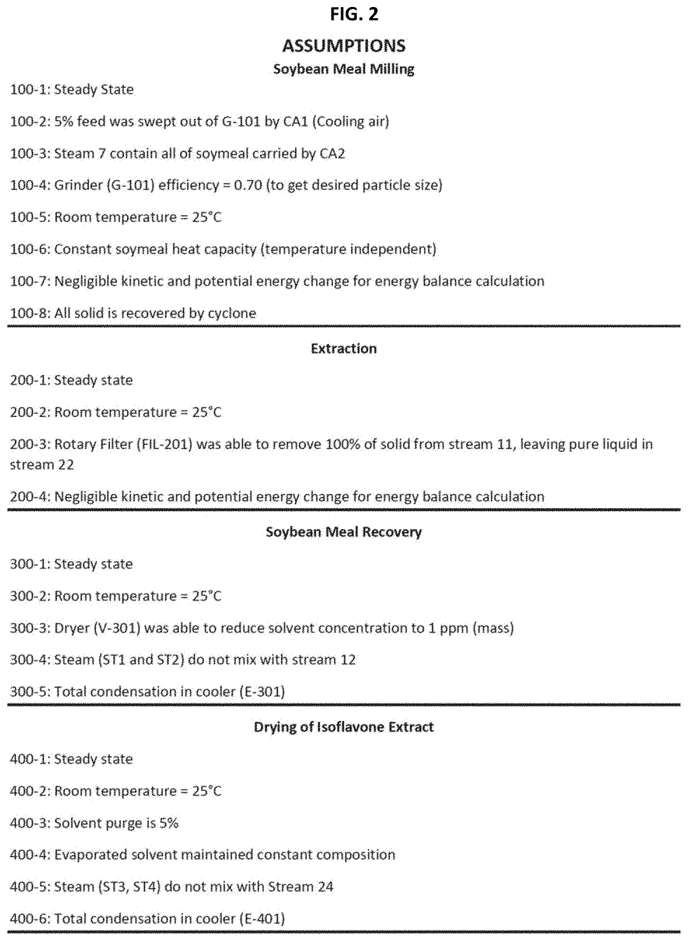

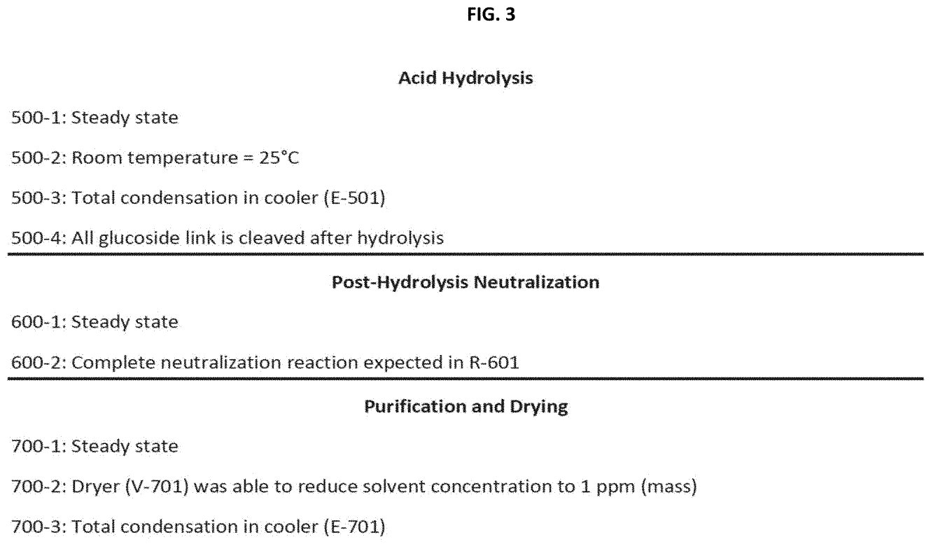

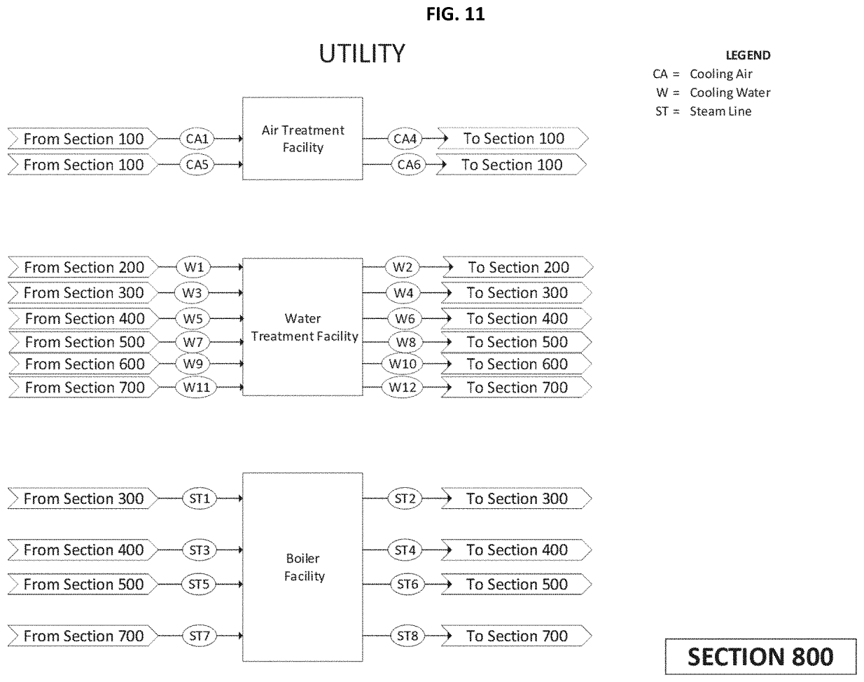

In some embodiments, one or more assumptions are made to determine the recommended system components (see FIG. 2 and FIG. 3). In some embodiments, the optimal system components are depicted in FIG. 4-FIG. 11.

In various embodiments, the soybean meal is suitable for commercial use after extraction of isoflavones. Commercial use includes any suitable agricultural or agrichemical use for soybean meal known in the art. Thus, the soybean meal extracted using the system described herein is not wasted and can be used for conventional commercial purposes. In various embodiments, the soybean meal after extraction is nutritionally suitable for animals. As used herein, “nutritionally suitable” means that the post-extraction soybean meal can be formulated into various animal feeds to provide the necessary and/or desired nutrient and/or protein content as suitable for the particular animal feed. In various embodiments, the animal feed is suitable for at least one animal selected from the group consisting of poultry, beef cattle, dairy cattle, swine, fish, dog, and cat. The post-extraction soybean meal is substantially free of one or more of the isoflavones described herein.

The system and methods described herein can extract about 0.1 kg/h to about 1 kg/h of isoflavones, including glycosylated isoflavones, isoflavone aglycones, or combinations thereof. In various embodiments, the system and methods described herein can extract about 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1 kg/h of isoflavones.

In various embodiments, the systems and methods described herein can be used to extract isoflavones from other raw materials such as, but not limited to, birch bark, chili pepper, and citrus peels.

### Methods of Soybean Meal Extraction

In another aspect, the present disclosure relates to a method for extracting one or more isoflavones from soybean meal. In one embodiment, the extraction of one or more isoflavones from soybeans is performed at a commercial scale. In some embodiments, the isoflavone is daidzein (7-hydroxy-3-(4-hydroxyphenyl)-4H-chromen-4-one), glycitein (7-Hydroxy-3-(4-hydroxyphenyl)-6-methoxy-4H-1-benzopyran-4-one), genistein (5,7-Dihydroxy-3-(4-hydroxyphenyl)-4H-1-benzopyran-4-one), or a combination thereof.

In various embodiments, the systems and methods described herein provide isoflavone aglycone composition containing genistein, glycitein, daidzein, or a mixture thereof.

| Isoflavone | Structure |
|---|---|
| Genistein | 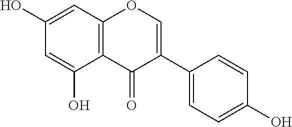 [SMILES: O=c1c(-c2ccc(O)cc2)coc2cc(O)cc(O)c12] |
| Daidzein | 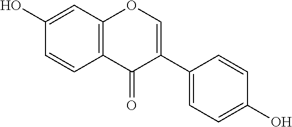 [SMILES: O=c1c(-c2ccc(O)cc2)coc2cc(O)ccc12] |
| Glycitein | 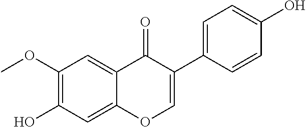 [SMILES: COc1cc2c(=O)c(-c3ccc(O)cc3)coc2cc1O] |

The systems and methods described herein can extract about 200 to about 2200 μg of daidzein per gram of soybean meal entering the extraction system. In various embodiments, the amount of daidzein extracted per gram of soybean meal is at least, greater than, or equal to about 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1020, 1040, 1060, 1080, 1100, 1120, 1140, 1160, 1180, 1200, 1220, 1240, 1260, 1280, 1300, 1320, 1340, 1360, 1380, 1400, 1420, 1440, 1460, 1480, 1500, 1520, 1540, 1560, 1580, 1600, 1620, 1640, 1660, 1680, 1700, 1720, 1740, 1760, 1780, 1800, 1820, 1840, 1860, 1880, 1900, 1920, 1940, 1960, 1980, 2000, 2020, 2040, 2060, 2080, 2100, 2120, 2140, 2160, 2180, or about 2200 μg.

The systems and methods described herein can extract about 200 to about 1500 μg of genistein per gram of soybean meal entering the extraction system. In various embodiments, the amount of genistein extracted per gram of soybean meal is at least, greater than, or equal to about 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1020, 1040, 1060, 1080, 1100, 1120, 1140, 1160, 1180, 1200, 1220, 1240, 1260, 1280, 1300, 1320, 1340, 1360, 1380, 1400, 1420, 1440, 1460, 1480, or about 1500 μg.

The systems and methods described herein can extract about 200 to about 2200 μg of glycitein per gram of soybean meal entering the extraction system. In various embodiments, the amount of glycitein extracted per gram of soybean meal is at least, greater than, or equal to about 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1020, 1040, 1060, 1080, 1100, 1120, 1140, 1160, 1180, 1200, 1220, 1240, 1260, 1280, 1300, 1320, 1340, 1360, 1380, 1400, 1420, 1440, 1460, 1480, 1500, 1520, 1540, 1560, 1580, 1600, 1620, 1640, 1660, 1680, 1700, 1720, 1740, 1760, 1780, 1800, 1820, 1840, 1860, 1880, 1900, 1920, 1940, 1960, 1980, 2000, 2020, 2040, 2060, 2080, 2100, 2120, 2140, 2160, 2180, or about 2200 μg.

In one embodiment, the method comprises extracting an isoflavone-glucoside from soybean meal, hydrolyzing the isoflavone glucoside to obtain an isoflavone aglycone, neutralizing the isoflavone aglycone, and purifying the isoflavone aglycone.

In some embodiments, the step of extracting an isoflavone-glucoside from soybean meal is preceded by the step of grinding the soybean meal (FIG. 1). The soybean meal can be ground to a particle size discussed elsewhere herein. In one embodiment, the soybean meal is ground to a size of about 25 μm to about 65 μm.

The step of extracting an isoflavone-glucoside from soybean meal can be performed using turbo-extraction (TE), maceration (MC), ultrasound-assisted extraction (UAE), or supercritical fluid extraction (SFE). In some embodiments, the isoflavone-glucoside is extracted from the soybean meal via contact with an organic solvent. In one embodiment, the organic solvent is ethanol. In some embodiments, the organic solvent is mixed with water.

In some embodiments, the step of extracting an isoflavone-glucoside is followed by the step of separating the soybean meal from the isoflavone-glucoside. The soybean meal can be separated from the isoflavone-glucoside using a solid-liquid separation method selected from sedimentation (SDM), filtration (FLT,1), and centrifugation (CNF). In some embodiments, the separated (i.e. recovered) soybean meal can be recycled back to the animal feed industry without disrupting the existing agricultural chain.

In some embodiments, the step of extracting an isoflavone-glucoside is followed by the step of recovering (DRY,1) the solvent used from the extraction step. In one embodiment, the solvent used in the extraction step can be recycled and in the extraction one or more isoflavones from soybeans.

The step of hydrolyzing the isoflavone glucoside to obtain an isoflavone aglycone comprises acid hydrolysis (AHY) of the isoflavone-glucoside to cleave the natural glucose attached to the isoflavone molecule. The acid hydrolysis can use any acid and concentration of acid known to a person of skill in the art. In one embodiment, a solution of hydrochloric acid is used for the acid hydrolysis. In one embodiment, the solution of hydrochloric acid has a molarity of between about 0.1 M and about 10 M. In one embodiment, the solution of hydrochloric acid has a molarity of between about 0.1 M and about 8 M. In one embodiment, the solution of hydrochloric acid has a molarity of between about 0.1 M and about 6 M. In one embodiment, the solution of hydrochloric acid has a molarity of between about 0.1 M and about 4 M. In one embodiment, the solution of hydrochloric acid has a molarity of between about 2 M and about 4 M.

The step of neutralizing the isoflavone aglycone comprises contacting a solution comprising an acid used for acid hydrolysis and the isoflavone aglycone with a base. The base can comprise a solution of any base at any concentration known to a person of skill in the art. In one embodiment, the base used for neutralization is sodium hydroxide. In one embodiment, the sodium hydroxide solution has a molarity of between about 0.1 M and about 10 M. In one embodiment, the sodium hydroxide solution has a molarity of between about 0.1 M and about 8 M. In one embodiment, the sodium hydroxide solution has a molarity of between about 0.1 M and about 6 M. In one embodiment, the sodium hydroxide solution has a molarity of between about 0.1 M and about 4 M. In one embodiment the sodium hydroxide solution has a molarity of between about 1 M and about 3 M. In one embodiment, a 2 M solution of sodium hydroxide in ethanol is used to neutralize the isoflavone aglycone. In some embodiments, the step of neutralizing the isoflavone aglycone further comprises the step of removing a precipitated salt formed during the neutralization reaction (FLT,2).

The step of purifying the isoflavone aglycone comprises crystallization (CRYS), organic solvent nanofiltration (OSN), or chromatography (CHRM) to remove remaining impurities from the isoflavone aglycone product. An antisolvent addition at a 5:1 mass ratio of water to isoflavone aglycones was specified. The equilibrium can be shifted to force nonpolar isoflavone to precipitate out of the solution by adding water. A filtration unit can be used to recover the purified powder. Organic solvent nanofiltration can also be used to separate the solvent from the valuable product with high efficiency. In some embodiments, the step of purifying the isoflavone aglycone further comprises the step of drying purified isoflavone aglycone (DRY,2) to remove traces of solvent from the final isoflavone aglycone product.

In some embodiments, General Algebraic Modeling Systems (GAMS) optimization is used to select the optimal process to extract an isoflavone-glucoside from soybean meal (i.e. TE, MC, UAE), the optimal process to separate the soybean meal from the isoflavone-glucoside (i.e. SDM, FLT,1, CNF), and the optimal process to purify the isoflavone aglycone (i.e. CRYS, OSN, CHRM). In one embodiment, GAMS optimization uses Mixed-Integer Nonlinear Programing (MINLP) wherein material and energy balances, design options, utilities, cost, and/or industrial constraints are considered to provide a recommended set of separation and purification technologies required to extract and isolate isoflavone aglycones from soybean meal at the commercial scale.

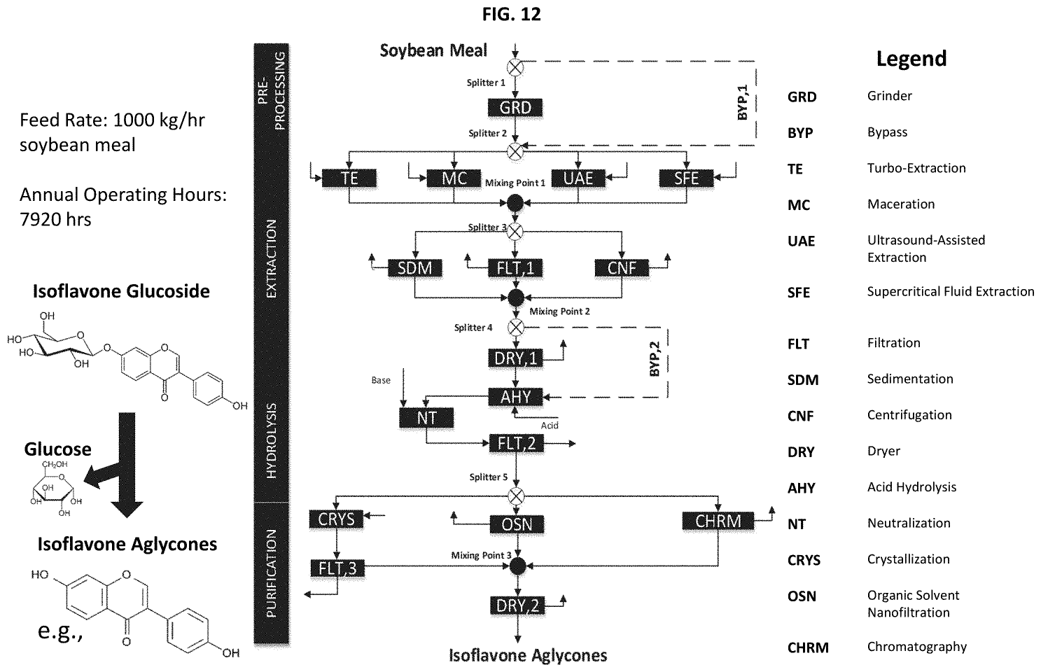

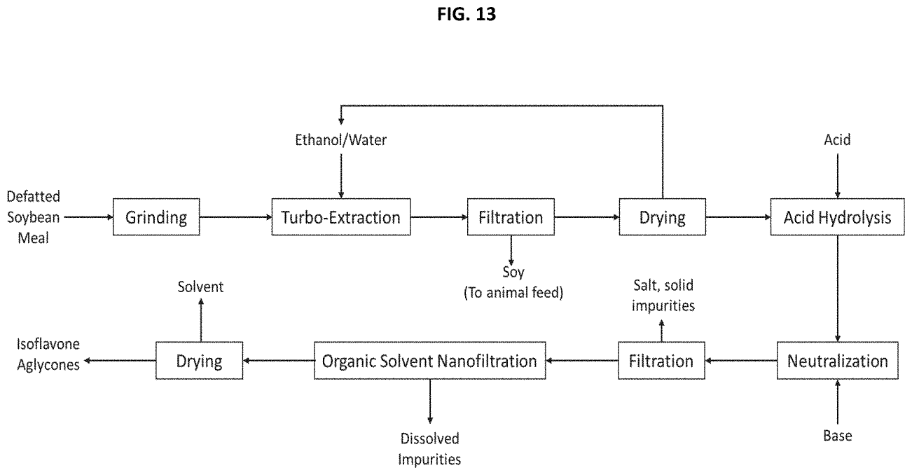

In some embodiments, the method for commercial extraction one or more isoflavones from soybean meal comprises the steps of grinding the soybean meal, turbo-extracting an isoflavone-glucoside from the soybean meal, separating the soybean meal from the isoflavone-glucoside, recovering a solvent used in the turbo-extraction, hydrolyzing the isoflavone glucoside to obtain an isoflavone aglycone, neutralizing the isoflavone aglycone, removing a precipitated salt formed during the neutralization reaction, nanofiltering the neutralized isoflavone aglycone, and drying the final isoflavone aglycone product (FIG. 12 and FIG. 13).

### EXPERIMENTAL EXAMPLES

The invention is further described in detail by reference to the following experimental examples. These examples are provided for purposes of illustration only, and are not intended to be limiting unless so specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Commercial Scale Isoflavone Extraction and Subsequent Conversion to Value-Added Chemicals and Materials

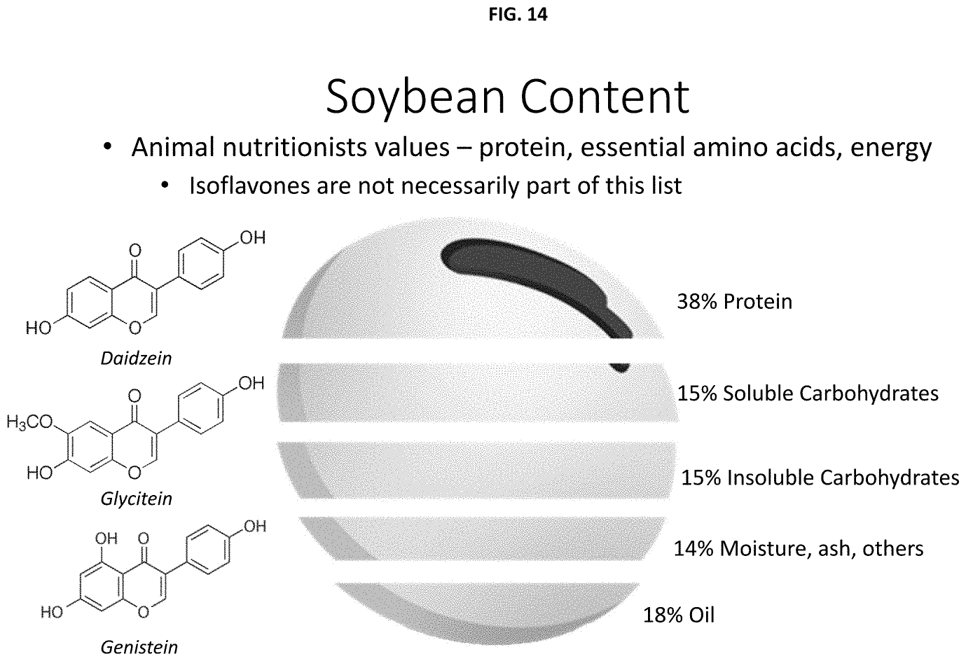

The present disclosure relates to a sustainable system and method for commercial scale extraction of isoflavones from soybean meal. Over 125 million metric tons of soybean is produced in the US annually, wherein a sizable portion is exported and much of the remaining soybean crop is used for animal feed. While the protein and carbohydrate content of soybeans make them useful for animal feed (FIG. 14), soybeans contain a small amount of isoflavones which can be used in the food, beverage, cosmetic, nutraceutical, or materials synthesis industries.

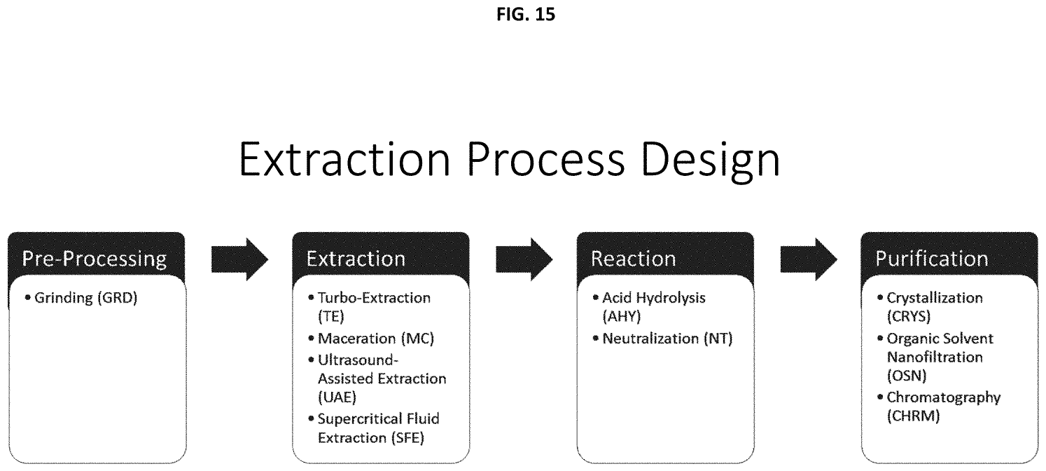

The present disclosure provides a method for the extraction of isoflavone that does not disrupt the current supply chain (FIG. 15). Specifically, the inventive method does not breakdown the soybean, which would render the original material unusable for animal feed and removes isoflavones, which are proven detrimental to the physiological functions of livestock upon excess consumption.

The extracted isoflavone can be further processed and purified using conventional means and can be then be used in other commercially viable pathways and products. The extracted isoflavones can be introduced into the bio-based polymers and composites market and can also be used to supplement the value-chain of isoflavone-containing commercial products such as dietary supplements, cosmetics, and nutraceuticals. The market of engineering plastics in North America was estimated at 81 billion (USD) in 2018 with projected growth to 115 billion (USD) by 2023. Although most of these materials are derived from petroleum, the market of bio-based polymers is projected to reach a compound annual growth rate of 11% between 2018 and 2023.

To date, petroleum-derived chemicals remain as the primary source for polymer production because of the inexpensive prices related to the acquisition of this raw material and the lack of political support for bio-based chemicals. Over 90% of epoxy resins manufactured today stemmed from Bisphenol A (BPA), which is an estrogen antagonist, endocrine disrupter, and a human carcinogen derived from petroleum. This chemical is favored among applications such as coatings, adhesives, composites, food containers, and plastic bags. The aromatic content and π-π stacking allow BPA-based materials to exhibit high thermal and structural stability and excellent mechanical properties. A suitable replacement for BPA is required for applications involving large amounts of human contact because of the potential of this substance to leach. Bio-based chemicals are a well-known alternative for petroleum-derived sources. One significant advantage associated with using bio-based materials is the reduction in greenhouse gas emission to the atmosphere because the reaction steps required to produce the molecule is not needed. However, the challenges associated with the successful implementation of bio-based materials are caused by the high price of raw material acquisition and sub-par performance in comparison to petroleum-based products. Recent advances have synthesized numerous bio-based materials with mechanical and thermal properties comparable to petroleum-derived materials. For instance, Lignin, a byproduct, and waste of the pulp and papermaking industry possess high thermal and structural properties and an abundance of hydroxyl groups for functionalization. Lignin derivatives such as vanillin, vanillyl alcohol, and guaiacol have also been used to produce novel polymer resins for adhesives, coating, flame retardant, high-performance, and composite applications. Alternatively, similar reaction chemistry and chemical modification can be performed on many other chemicals derived from renewable resources, including soybean, betulin, citrus peels, and other underutilized resources. Soybean isoflavones have also been used to synthesize high-performance materials with strong thermal resistance. Despite the favorable properties imparted by soy isoflavones, the extractions of this material have only been optimized at lab-scale with consideration for only one major processing pathway.

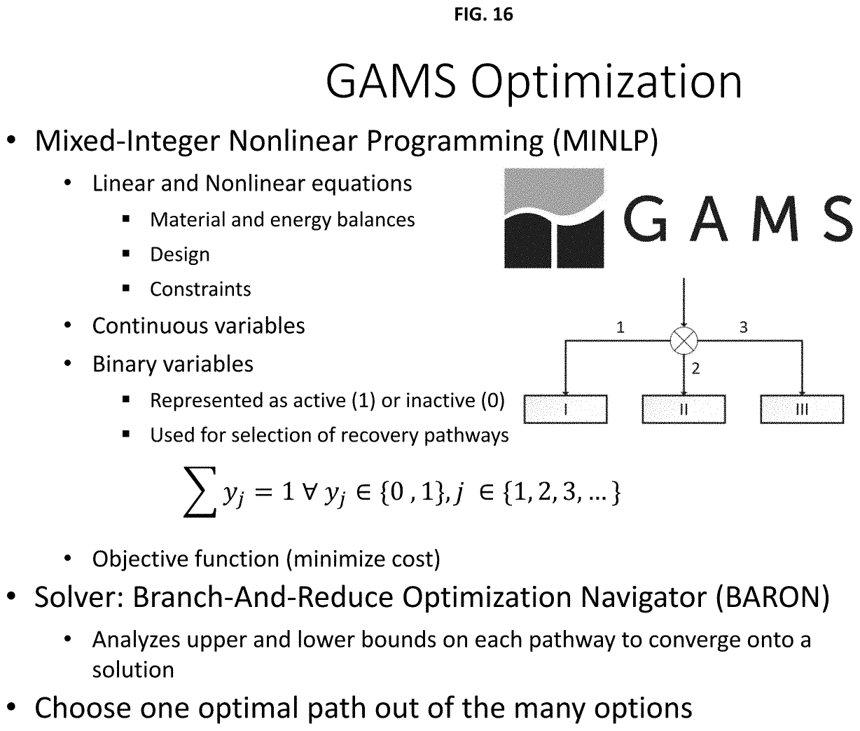

Therefore, the present disclosure presents a superstructure-based analysis of commercial-scale soy isoflavones extraction to address the increase in isoflavone demands. This analysis simultaneously compared the alternative extraction and purification technologies to assess the most economically viable pathway in the General Algebraic Modeling Systems (GAMS; FIG. 16). The analysis of each model considered material and energy balances, design options, utilities, cost, and industrial constraints, to provide a recommended set of separation and purification technologies required to extract and isolate isoflavone aglycones from soybean meal at the commercial scale. By analyzing alternative options simultaneously, this analysis can systematically assess the viability of the bio-based chemical extractions industry.

In FIG. 1, a superstructure-based optimization framework for designing a commercial-scale soy isoflavones extraction process is presented. This framework includes mathematical models for various separation technologies with details involving mass and energy balances, equipment design, and cost. Four essential stages of acquiring purified isoflavone from soybean meal were considered, including pre-processing, extraction, acid hydrolysis, and purification. Pre-processing is used for particle-size reduction for enhanced product dissolution in the extraction stage. The isoflavone extraction superstructure considers the possibility of grinding (GRD) the soybeans to form a soybean meal as a pre-processing step. The extraction stage generally includes turbo-extraction (TE), maceration (MC), ultrasound-assisted extraction (UAE), and supercritical fluid extraction (SFE) for extracting isoflavone-glucosides from defatted soybean meal.

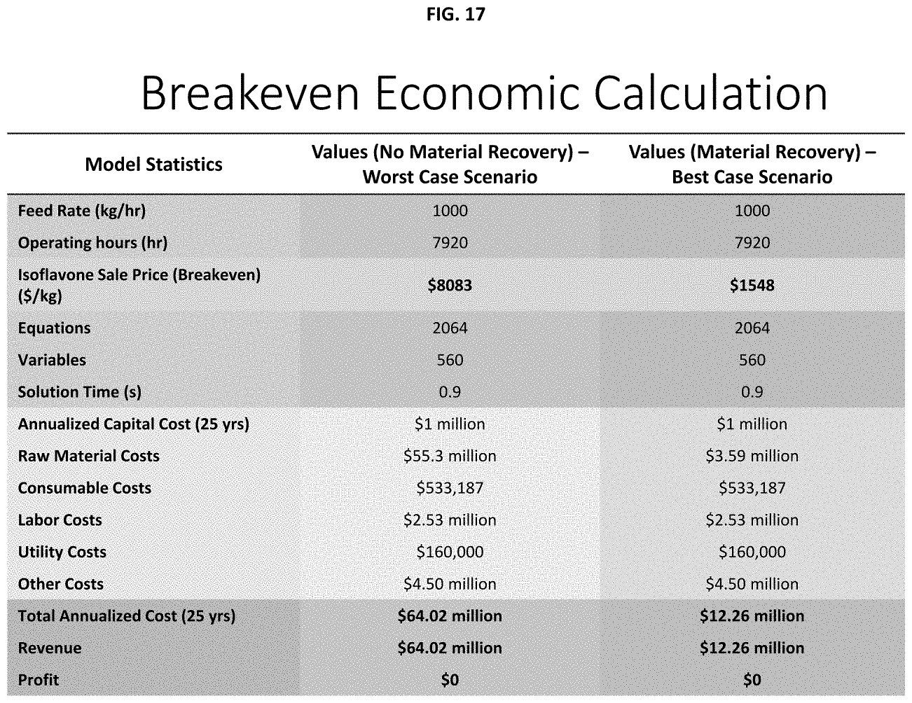

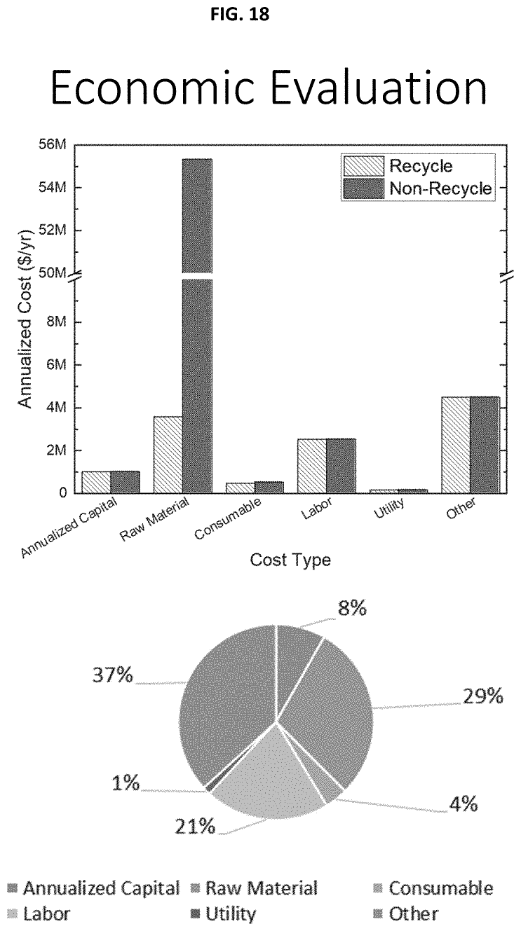

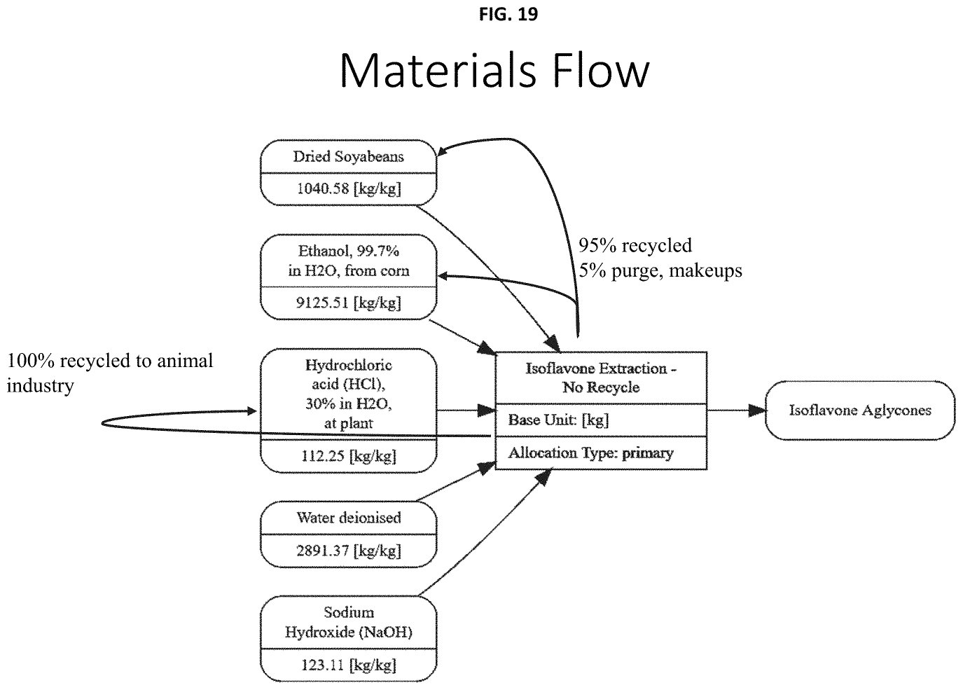

An aqueous alcohol mixture of ethanol and water was selected as the standard solvent configuration for TE, MC, and UAE. In SFE, ethanol was chosen as the polar co-solvent with supercritical carbon dioxide to facilitate the extraction. A conventional method such as Soxhlet extraction was excluded from consideration because this process is energy-intensive and have not been deemed viable at the commercial scale. Three solid-liquid separation methods, such as sedimentation (SDM), filtration (FLT,1), and centrifugation (CNF), were used to simulate soybean meal recovery after extraction. The recovered soybean meal can be recycled back to the animal feed industry without disrupting the existing agricultural chain. A drying (DRY) step was used to recover the solvents used from the extraction step. The isoflavone-glucoside extracted using the solvent-based methods is then subjected acid hydrolysis (AHY), which effectively cleaves the natural glucose attached to the isoflavone molecule. AHY is followed by the subsequent neutralization (N.T.) and filtration (FLT,2). In the purification stage, crystallization (CRYS), organic solvent nanofiltration (OSN), and chromatography (CHRM) were considered to remove the remaining impurities from the product. A final drying (DRY,2) was used to remove traces of solvent from the final isoflavone aglycone product. The analysis of each model considered material and energy balances, utilities, design options, industrial constraints, and costs (FIG. 17, FIG. 18, and FIG. 19).

The optimized extraction pathway was also deemed to be sustainable through an environmental impact analysis by the Sustainable Process Index (SPI). By analyzing alternative extraction options simultaneously, soy isoflavone extraction at the commercial-scale can be reasonably implemented to increase the availability of bio-based raw materials for meeting the current demands and promote the synthesis of useful chemicals and materials that are derived from renewable sources.

Before selecting the most optimal pathway out of the options presented, the mathematical model was built assuming a soybean meal composition for the initial process feed has been estimated to contain 97.85% insoluble solids, 2% impurities, and 0.15% isoflavone glucoside, wherein the insoluble solids contain protein, ash, starch, and fiber and the impurities represent fat and moisture that are considered soluble in conventional organic and aqueous solvents. The soybean meal feed flow rate of 1000 kg/hr with a yearly operating hour of 7920 hrs or 330 workdays was used. The soybean meal is first subjected to a grinding operation for particle size reduction from 65 μm to 25 μm. The grinding model efficiency was assumed to be 75%. Cooling air was introduced to minimize soy thermal degradation.

In the extraction stage, four common extracting options were considered, including turbo-extraction, maceration, ultrasound-assisted extraction, and supercritical fluid extraction. In turbo-extraction, an agitator power of 1.75 kW/m³ was used based on the average fluid properties. Cooling water is required to offset the temperature increase resulting from the high shear produced by this mixing process. In maceration, time is the major parameter that strongly dictates the extraction efficiency. Agitation is not required because the solid soy residence time in the extracting solution is much longer than turbo-extraction. In ultrasound-assisted extraction, an 80% efficiency and residence time of 15 min were chosen based on previous experimental findings. Based on existing research on isoflavone extraction through supercritical fluid extraction, a ratio of 0.0000863 kg isoflavone/kg CO₂ required was used for establishing the optimal extraction condition.

Following the solvent-based extraction steps, three choices of solid separation, such as sedimentation, filtration, and centrifugation, were considered. In sedimentation, 75% efficiency was assumed with a final liquid purity of 99% and a residence time of 30 min. For filtration, the flux of the membrane was specified as 0.2 kg/m²*s. The retention factor condition was set to recover 99% of the isoflavone glucoside and solvent while removing unwanted solids from the product stream. In centrifugation, the solid-liquid separation efficiency was set to 95%. A freeze dryer model was implemented after the solid separation stage to remove 99% of the solvents, assuming an average heat transfer coefficient of 180 kJ/(m²*K*hr). The dried isoflavone glucoside is hydrolyzed through acid hydrolysis to obtain isoflavone aglycones. The amount of acid (hydrochloric acid) required was specified as 0.791 kg HCl/kg solution to reach the ideal concentration based on the optimized experimental conditions. Steam at 120° C. was selected as the heating agent to provide sufficient energy for hydrolysis. Sodium hydroxide was chosen as the base at the theoretical molar equivalent to hydrochloric acid for neutralizing excess acid after the hydrolysis reaction. A filtration unit (FLT2) was used following the neutralization reaction to remove excess salt that precipitated from the acid-base reaction in an organic mixture.

Three alternative routes for removing any remaining impurities from the product stream were considered in the purification stage. An antisolvent addition at a 5:1 mass ratio of water to isoflavone aglycones was specified. The equilibrium can be shifted to force nonpolar isoflavone to precipitate out of the solution by adding water. A filtration unit can be used to recover the purified powder. Organic solvent nanofiltration can also be used to separate the solvent from the valuable product with high efficiency. The third purification option requires chromatography. Several design variables of a chromatography column have been specified as parameters to reduce the overall complexity. These parameters include the chromatography column's width being 0.05 m, the theoretical plate height being 0.0035, and the ratio between the column length and diameter being 0.14.

The superstructure-based optimization approach determined that an initial particle size reduction (GRD) is required to enhance the extraction efficiency. A high-turbulence mixing tank (TE) with the addition of green solvents such as ethanol and water can be used to extract isoflavone glucosides from the soybean meal. The remaining soybean meal can be recovered, dried, and used in the animal feed industry because this extraction process does not adversely affect the protein content and other essential nutrients. Acid hydrolysis and subsequent neutralization can be used to cleave the glucoside link and obtain the final product in the solution phase. A membrane process such as organic solvent nanofiltration (OSN) can be used to improve the purity of the final isoflavone aglycone concentrate. A final drying step removes traces of liquid solvent from the product to a negligible level. This pathway is represented in FIG. 12 and FIG. 13. The disclosed isoflavone extraction pathway is economically feasible with minimal environmental impacts based on 1000 kg/hr feed of soybean meal.

Based on cost annualization over a 25-year period, the cost required to maintain an isoflavone extraction process with 1000 kg/hr soybean meal feed is approximately $12.26 million annually. The cost distribution for the optimal extraction pathway is shown in FIG. 22. Overhead cost (Other) accounts for 37% of the total annual process cost, followed by raw material (29%), labor (21%), capital cost (8%), consumable (4%), and utility (1%). The cost of purchasing defatted soybean meal is $0.45/kg, based on soybean meal commodity price average between 2010-2020. The price of solvent and other consumables are taken from SuperPro Designer databanks. There is a ±40% uncertainty associated with the calculated price because the model equations used to solve the optimization problem are based on shortcut methods. The uncertainty can be further reduced through detailed designs and modeling tools such as Aspen Plus and SuperPro Designer.

An environmental impact assessment was completed using the Sustainability Process Index (SPI) to assess the ecological footprint of the optimal process. To extract isoflavone from soybean meal, the material inputs include ethanol, water, soybean, hydrochloric acid, and sodium hydroxide. The SPI database standardized the potential emissions to water, soil, and air and the impacts on the Earth's resources as m² of arable land area. The ecological footprint of the isoflavone extraction process equates to 87,204 m²·a/kg isoflavone produced. Based on a yearly operation hour of 7920 hrs, the theoretical annual arable land area required to maintain process sustainability equates to 690 million m²·a/yr. The Earth's land surface area equates to 149 trillion m² (57.51 million mi²). As of 2016, the arable land was estimated to be 11.06% of the total land surface area (16.5 trillion m²). The ecological footprint of the isoflavone extraction accounts for 0.004% of the total arable area on Earth annually. It should be noted that the arable land area required is a theoretical representation of the process sustainability. It does not reflect on the actual land area required to operate the process.

Future work will entail the consideration of other extraction, solid separation, evaporation, and purification technologies to expand the solution search space beyond the existing superstructure. The price of raw materials, products (isoflavone aglycones), equipment, labor, overhead, consumables, and utility, can vary according to the operation region. At this time, the values for estimating the cost of the commercial extraction were done according to the market average and design recommendations from Chemical Engineering design texts. Additionally, the extracted isoflavones have characteristics that are desirable in polymer design and performance and will be incorporated into biopolymers that may provide an economically viable replacement for petroleum-derived materials.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the disclosure. The appended claims are intended to be construed to include all such embodiments and equivalent variations.

### ENUMERATED EMBODIMENTS

The following enumerated embodiments are provided, the numbering of which is not to be construed as designating levels of importance:

Embodiment 1 provides system for commercial scale extraction of an isoflavone from soybean meal, the system comprising: an optional grinder, an extraction component, a solid-liquid separation component, an acid hydrolysis component, a neutralization component, and a purification component,

wherein the system extracts about 500 to about 1000 kg//h of soybean meal.

Embodiment 2 provides the system of embodiment 1, wherein the extraction component is selected from a turbo-extraction (TE) component, a maceration (MC) component, an ultrasound-assisted extraction (UAE) component, or supercritical fluid extraction (SFE) component.

Embodiment 3 provides the system of any one of embodiments 1-2, wherein the solid-liquid separation component is selected from a sedimentation (SDM) component, a filtration (FLT,1) component, and a centrifugation (CNF) component.

Embodiment 4 provides the system of any one of embodiments 1-3, wherein the purification component is selected from a crystallization (CRYS) component, an organic solvent nanofiltration (OSN) component, or a chromatography (CHRM) component.

Embodiment 5 provides the system of any one of embodiments 1-4, wherein solid-liquid separation component further comprises a recovery component to recover one or more extraction solvents used in the extraction component.

Embodiment 6 provides the system of any one of embodiments 1-5, wherein the neutralization component further comprises a filtration (FLT,2) component to remove precipitated salt formed during a neutralization reaction which occurs in the neutralization component.

Embodiment 7 provides the system of any one of embodiments 1-6, wherein the soybean meal is suitable for commercial use after extraction of isoflavones.

Embodiment 8 provides the system of any one of embodiments 1-7, wherein the soybean meal after extraction is nutritionally suitable for an animal.

Embodiment 9 provides the system of any one of embodiments 1-8, wherein the animal is at least one selected from the group consisting of poultry, beef cattle, dairy cattle, swine, fish, dog, and cat.

Embodiment 10 provides the system of any one of embodiments 1-9, wherein the isoflavone comprises genistein, daidzein, glycitein, glycosylated genistein, glycosylated daidzein, glycosylated glycitein, or combinations thereof.

Embodiment 11 provides the system of any one of embodiments 1-10, wherein the isoflavone comprises 200 to about 2200 μg of daidzein per gram of soybean meal used in the extraction component, 200 to about 2200 μg of glycitein per gram of soybean meal used in the extraction component, about 200 to about 1500 μg of genistein per gram of soybean meal used in the extraction component, or combinations thereof.

Embodiment 12 provides the system of any one of embodiments 1-11, wherein the system produces about 0.1 to about 1.0 kg/h of isoflavones.

Embodiment 13 provides a method of commercial scale extraction of one or more isoflavones from soybean meal, comprising:

extracting an isoflavone-glucoside from soybean meal at a soybean meal feed rate of about 500 to about 1000 kg//h,

hydrolyzing the isoflavone glucoside to obtain an isoflavone aglycone,

neutralizing the isoflavone aglycone, and

purifying the isoflavone aglycone.

Embodiment 14 provides the method of embodiment 13, wherein the step of extracting the isoflavone-glucoside from the soybean meal comprises turbo-extraction (TE), maceration (MC), ultrasound-assisted extraction (UAE), or supercritical fluid extraction (SFE).

Embodiment 15 provides the method of any one of embodiments 13-14, wherein the step of purifying the isoflavone aglycone comprises crystallization (CRYS), organic solvent nanofiltration (OSN), or chromatography (CHRM).

Embodiment 16 provides the method of any one of embodiments 13-15, wherein the step of extracting an isoflavone-glucoside from soybean meal is preceded by the step of grinding the soybean meal.

Embodiment 17 provides the method of any one of embodiments 13-16, wherein the step of extracting the isoflavone-glucoside is followed by the step of separating the soybean meal from the isoflavone-glucoside.

Embodiment 18 provides the method of any one of embodiments 13-17, wherein the step of neutralizing the isoflavone aglycone is followed by the step of removing a precipitated salt formed during neutralization of the isoflavone aglycone.

Embodiment 19 provides the method of any one of embodiments 13-18, wherein the soybean meal is suitable for commercial use after extraction of isoflavones.

Embodiment 20 provides the method of any one of embodiments 13-19, wherein the soybean meal after extraction is nutritionally suitable for animals.

Embodiment 21 provides the method of any one of embodiments 13-20, wherein the animal is at least one selected from the group consisting of poultry, beef cattle, dairy cattle, swine, fish, dog, and cat.

Embodiment 22 provides the method of any one of embodiments 13-21, wherein the isoflavones comprise genistein, daidzein, glycitein, glycosylated genistein, glycosylated daidzein, glycosylated glycitein, or combinations thereof.

Embodiment 23 provides the method of any one of embodiments 13-22, comprising providing an isoflavone glucoside or isoflavone aglycone extract comprising 200 to about 2200 μg of daidzein per gram of soybean meal used in the extraction, 200 to about 2200 μg of glycitein per gram of soybean meal used in the extraction, about 200 to about 1500 μg of genistein per gram of soybean meal used in the extractions, or combinations thereof.

Embodiment 24 provides the method of any one of embodiments 13-23, wherein the isoflavone aglycone is provided at a rate of about 0.1 to about 1.0 kg/h.

What is claimed is:

1. A system for commercial scale extraction of an isoflavone from soybean meal, the system comprising:
an optional grinder,
an extraction component,
a solid-liquid separation component,
an acid hydrolysis component,
a neutralization component, and
a purification component,
wherein the system extracts about 500 to about 1000 kg//h of soybean meal.

2. The system of claim 1, wherein the extraction component is selected from a turbo-extraction (TE) component, a maceration (MC) component, an ultrasound-assisted extraction (UAE) component, or supercritical fluid extraction (SFE) component.

3. The system of claim 1, wherein the solid-liquid separation component is selected from a sedimentation (SDM) component, a filtration (FLT,1) component, and a centrifugation (CNF) component.

4. The system of claim 1, wherein the purification component is selected from a crystallization (CRYS) component, an organic solvent nanofiltration (OSN) component, or a chromatography (CHRM) component.

5. The system of claim 3, wherein solid-liquid separation component further comprises a recovery component to recover one or more extraction solvents used in the extraction component.

6. The system of claim 1, wherein the neutralization component further comprises a filtration (FLT,2) component to remove precipitated salt formed during a neutralization reaction which occurs in the neutralization component.

7. The system of claim 1, wherein at least one of the following applies:
the soybean meal is suitable for commercial use after extraction of isoflavones;
the soybean meal after extraction is nutritionally suitable for an animal.

8. The system of claim 7, wherein the animal is at least one selected from the group consisting of poultry, beef cattle, dairy cattle, swine, fish, dog, and cat.

9. The system of claim 1, wherein the isoflavone comprises genistein, daidzein, glycitein, glycosylated genistein, glycosylated daidzein, glycosylated glycitein, or combinations thereof.

10. The system of claim 9, wherein the isoflavone comprises 200 to about 2200 μg of daidzein per gram of soybean meal used in the extraction component, 200 to about 2200 μg of glycitein per gram of soybean meal used in the extraction component, about 200 to about 1500 μg of genistein per gram of soybean meal used in the extraction component, or combinations thereof.

11. The system of claim 1, wherein the system produces about 0.1 to about 1.0 kg/h of isoflavones.

12. A method of commercial scale extraction of one or more isoflavones from soybean meal, the method comprising:
extracting an isoflavone-glucoside from soybean meal at a soybean meal feed rate of about 500 to about 1000 kg//h,
hydrolyzing the isoflavone glucoside to obtain an isoflavone aglycone,
neutralizing the isoflavone aglycone, and
purifying the isoflavone aglycone.

13. The method of claim 12, wherein the step of extracting the isoflavone-glucoside from the soybean meal comprises turbo-extraction (TE), maceration (MC), ultrasound-assisted extraction (UAE), or supercritical fluid extraction (SFE).

14. The method of claim 12, wherein the step of purifying the isoflavone aglycone comprises crystallization (CRYS), organic solvent nanofiltration (OSN), or chromatography (CHRM).

15. The method of claim 12, wherein at least one of the following applies:
the step of extracting an isoflavone-glucoside from soybean meal is preceded by the step of grinding the soybean meal;
the step of extracting the isoflavone-glucoside is followed by the step of separating the soybean meal from the isoflavone-glucoside.

16. The method of claim 12, wherein the step of neutralizing the isoflavone aglycone is followed by the step of removing a precipitated salt formed during neutralization of the isoflavone aglycone.

17. The method of claim 12, wherein at least one of the following applies:
the soybean meal is suitable for commercial use after extraction of isoflavones;
the soybean meal after extraction is nutritionally suitable for animals.

18. The method of claim 17, wherein the animal is at least one selected from the group consisting of poultry, beef cattle, dairy cattle, swine, fish, dog, and cat.

19. The method of claim 12, wherein the isoflavones comprise genistein, daidzein, glycitein, glycosylated genistein, glycosylated daidzein, glycosylated glycitein, or combinations thereof.

20. The method of claim 12, wherein at least one of the following applies:
an isoflavone glucoside or isoflavone aglycone extract is provided comprising 200 to about 2200 μg of daidzein per gram of soybean meal used in the extraction, 200 to about 2200 μg of glycitein per gram of soybean meal used in the extraction, about 200 to about 1500 μg of genistein per gram of soybean meal used in the extractions, or combinations thereof;
the isoflavone aglycone is provided at a rate of about 0.1 to about 1.0 kg/h.
